(19) United States
(12) Patent Application Publication
(10) Pub. No.: US 2020/0118465 A1
(43) Pub. Date: Apr. 16, 2020

(54) Laser Line Directional System for 3D Anatomy Ultrasound Phantom Trainer

(71) Applicant: UNIVERSITY OF SOUTH CAROLINA, COLUMBIA, SC (US)

(72) Inventors: RICHARD HOPPMANN, COLUMBIA, SC (US); JOHN EBERTH, COLUMBIA, SC (US); BROOKS LANE, COLUMBIA, SC (US); TOUFIC ROBERT HADDAD, COLUMBIA, SC (US); MICHAEL SHAUN RIFFLE, SWANSEA, SC (US)

(21) Appl. No.: 16/604,190

(22) PCT Filed: Mar. 22, 2018
(86) PCT No.: PCT/US18/23748

§ 371 Date: Oct. 10, 2019

Related U.S. Application Data
(60) Provisional application No. 62/486,107, filed on Apr. 17, 2017.
(60) Provisional application No. 62/632,166, filed on Feb. 19, 2018.

(51) Int. Cl. G09B 23/28; A61B 8/00; G16H 50/50

(52) U.S. Cl. CPC G09B 23/286; A61B 8/469; G16H 50/50; A61B 8/4254

(57) ABSTRACT

The skill of performing ultrasound is becoming a standard in medical education and clinical practice across a wide range of disciplines and clinical practices. Ultrasound is being used as a clinical tool by physicians, nurses, and other healthcare providers. A major limitation to the broad incorporation of ultrasound is the lack of qualified users and instructors. Simple and effective methods to teach the many new learners of ultrasound scanning are needed. Presently disclosed subject matter uses a visible color laser beam originating from an ultrasound probe itself or from a laser light pointer attached to an ultrasound probe which can penetrate a clear-gel phantom. The laser light is aligned with the direction of flow of the invisible ultrasound waves so that the learner will know where the ultrasound waves are hitting the anatomical target within the phantom gel. Immediate visual feedback from the laser light informs the learner on how small movements of the probe affect the direction of the ultrasound waves and the quality of ultrasound image obtained, and allows an instructor to point out various aspects of anatomic structures. Using laser light helps learners more easily acquire the skill necessary to use ultrasound to guide catheters or needles to blood vessels or joint spaces to place a catheter, withdraw fluid, or inject medication.

### PRIORITY INFORMATION

The present application claims priority to U.S. Provisional Patent Application Ser. No. 62/486,107 titled “Laser Line Directional System for 3D Anatomy Ultrasound Phantom Trainer” by Hoppmann filed on Apr. 17, 2017, and claims priority to U.S. Provisional Patent Application Ser. No. 62/632,166 titled “Laser Line Directional System for 3D Anatomy Ultrasound Phantom Trainer” by Hoppmann et al. filed on Feb. 19, 2018, the disclosures of which are fully incorporated by reference herein and for all purposes.

### STATEMENT REGARDING FEDERALLY SPONSORED RESEARCH or DEVELOPMENT

The presently disclosed subject matter was made without government support.

### BACKGROUND OF THE PRESENTLY DISCLOSED SUBJECT MATTER

The skill of performing ultrasound is becoming a standard in medical education and clinical practice across a wide range of disciplines such as anatomy and pathology and clinical practice from primary care to orthopedics and neurosurgery. Ultrasound is being used as a clinical tool by physicians, nurses, physician assistants, emergency medicine technicians, midwives, medics, and other healthcare providers. Portable ultrasound is also being used by teachers in primary, secondary, graduate, and post-graduate education to teach life sciences.

A major limitation to the incorporation of ultrasound as a powerful teaching and clinical tool that can improve quality of medical care, improve patient safety, decrease healthcare cost with increasing access to important healthcare technology is the lack of qualified users and instructors. Simple and effective methods to teach the many new learners of ultrasound scanning are needed.

With so many new learners to ultrasound needed, it is essential that methods be developed to assist in their training. One method that has been developed is the use of phantom models of human tissue for learners to scan with ultrasound to improve their skills in obtaining a quality ultrasound image while gaining an appreciation of anatomy and pathology. Phantoms are also used to help learners develop skill in using ultrasound to guide a needle or catheter to a particular internal structure such as a blood vessel or joint space. The phantom model is embedded in a gel that allows ultrasound waves to penetrate the gel and reach the target tissue structure to produce an ultrasound image similar to that obtained with live models.

While there are some manufactures of ultrasound simulation that use computer generated images and simulated probes for teaching ultrasound skill, such systems do not use ultrasound waves. There are also simulation manufactures that use ultrasound waves to scan objects that produce ultrasound images that look similar to human tissue. However, such simulators do not show the direction of the ultrasound waves with laser light or any other method.

There are some 3D printing companies that print anatomical structures but they are not used in ultrasound training like the presently disclosed subject matter.

The market of ultrasound learners is expanding as indicated by the increasing number and variety of healthcare providers that are incorporating ultrasound into their education curricula and practice. Learners of ultrasound would total into the hundreds of thousands globally and the numbers of education centers particularly interested in the presently disclosed subject matter would be in the thousands. The use of ultrasound has extended far beyond the traditional users of radiologists, cardiologists, obstetricians and sonographers, to include virtually every healthcare provider at every level of practice. Portable ultrasound is becoming the stethoscope of the 21st century and everyone today using a stethoscope will likely be using portable ultrasound in the not too distant future. Such practitioners on a global level include nurses, physicians, medics, emergency medical technicians, acute disaster teams, physician assistants, and similar.

As a teaching tool for the life sciences, there would be a huge market of life science teachers from primary schools through graduate schools that would benefit from the presently disclosed technology.

### BRIEF DESCRIPTION OF THE PRESENTLY DISCLOSED SUBJECT MATTER

Aspects and advantages of the presently disclosed subject matter will be set forth in part in the following description, or may be apparent from the description, or may be learned through practice of the presently disclosed subject matter.

In general, it is a present object to provide improved ultrasound training arrangements, and associated methodology. It is a more particular object, in some instances, to provide an improved ultrasound device for use with a phantom.

The presently disclosed subject matter preferably will use a visible color laser beam originating from an ultrasound probe itself or from a laser light pointer attached to an ultrasound probe which can penetrate a clear-gel phantom. The laser light preferably will be aligned with the direction of flow of the invisible ultrasound waves so that the learner will know where the ultrasound waves are hitting the anatomical target within the phantom gel.

The immediate visual feedback from the laser light will inform the learner on how small movements of the probe affect the direction of the ultrasound waves and the quality of ultrasound image obtained thus enhancing the learners scanning skill. The laser beam will also allow an instructor to point out various aspects of the anatomic structures as the laser light strikes them. Thus, the learner is not only acquiring ultrasound skill but is also learning anatomy.

Using the laser light to give feedback as to the direction of the ultrasound waves can also be used to help learners more easily acquire the skill necessary to use ultrasound to guide catheters or needles to blood vessels or joint spaces to place a catheter, withdraw fluid, or inject medication. Learners of any procedure that uses ultrasound guidance such as thoracentesis, paracentesis, and tissue biopsies can use the laser light feedback to help acquire the necessary ultrasound skill to perform these procedures. It can also help them learn the appropriate anatomical location of where to insert the needle or catheter.

The presently disclosed ultrasound probe with laser light indicating the exact direction and anatomy contact points of the ultrasound waves can be used to teach all new users of ultrasound. It will provide immediate visual feedback and help the learner develop the manual dexterity and fine motor control of the ultrasound probe important to ultrasound scanning and capturing the best ultrasound images. The presently disclosed subject matter will enhance learning of ultrasound scanning and because of its simple and straightforward design can minimize the need for extensive direct supervision in training.

Learning with a probe that has a built-in laser line or one attached to the probe that the learner will ultimately be using clinically will also help with transfer of the learned ultrasound skill better than simulated ultrasound probes and ultrasound machines.

One presently disclosed exemplary embodiment relates to a method for training an operator to use an ultrasound device. Such method preferably comprises providing an ultrasound device having a probe which can be manipulated by an operator relative to a practice target, with such probe selectively projecting ultrasound waves; and associating with such probe a guide light device configured to project visible light in a projection area which coincides with that of ultrasound waves projected from the probe. With such an arrangement and methodology, an operator can manipulate the probe for ultrasonic scanning of a practice target aided by visually observing the illumination of the practice target by the projected visible light.

More particularly, for some embodiments of such method, such guide light device may project visible color laser light, aligned with the direction of invisible ultrasound waves. For some such embodiments, such guide light device may comprise a line laser built in to such ultrasound device probe.

In yet others, such guide light device may comprise a line laser attached to such ultrasound device probe. In some such instances, such line laser may be attached to such ultrasound device probe using a flexible elastic band holder conforming to the shape of such ultrasound device probe.

In still other variations of the foregoing methodology, such method may further comprise using 3D printing to produce a laser holder for attachment of such line laser to such ultrasound device probe.

In yet other alternatives, such practice target may comprise a phantom model embedded in a gel material. In some such instances, such phantom model may represent human anatomy-like structures; and such gel material may be transparent to light. In other such variations, such human anatomy-like structures may comprise one of bone, joint, and latex tubing for a blood vessel. For others, such human anatomy-like structures may comprise one of human tissue bone, joint, vessels, blood, fat, muscle, tendon, nerves, skin and organs.

In still other variations of such methodology, such phantom model may comprise one of replicas of normal anatomical structures, actual pathological specimens, and 3D replicas of pathological specimens, to facilitate training operators in how to identify pathology in the structures.

In yet others, such practice target may comprise a phantom model of at least one of anatomical and non-anatomical structures embedded in a gel material. For some such variations, such non-anatomical structures may comprise one of selected geometrical shapes of selected colors inserted into a gel for the operator being trained to practice scanning. For others, such structures embedded in a gel material may be one of rigid materials fully reflecting ultrasound waves without penetration or gel-like material of variable density and impedance that reflect a portion of the ultrasound waves to give an identifiable ultrasound image with a portion of the ultrasound waves to penetrate beyond the structure to allow deeper structures to also reflect the waves to give an ultrasound image effect similar to that in human tissue.

Yet another exemplary embodiment of presently disclosed subject matter may relate to a method of operator training, using an ultrasound device. Such method may preferably comprise providing an ultrasound device having an associated screen visible to an operator; and associating a guide light with the ultrasound device so that the operator can visually observe light indicating the exact direction and anatomy of contact points of ultrasound waves emanating from such ultrasound device.

For some such exemplary methods, such method may further comprise associating a practice target with such ultrasound device, such that the operator can manipulate the ultrasound device for ultrasonic scanning of such practice target aided by visually observing the illumination of the practice target by the projected visible light. In some such embodiments, such projected visible light may comprise laser light from a laser device associated with such ultrasound device. For others, such ultrasound device may have a probe manipulated by an operator; and such probe may have a laser light formed therewith or attached thereto. In some such instances, such laser light may comprise the output of a line laser.

It is to be understood that the presently disclosed subject matter equally relates to associated and/or corresponding device subject matter as well as the referenced presently disclosed methodologies. Yet another exemplary embodiment of presently disclosed subject matter relates to an ultrasound device with directional light for 3D anatomy ultrasound phantom trainer, comprising an ultrasound device and a guide light device combination. More specifically, such ultrasound device may preferably have a probe which can be manipulated by an operator relative to a practice target, with such probe selectively projecting ultrasound waves; and such guide light device associated with such probe, may be preferably configured to project visible light in a projection area which coincides with that of ultrasound waves projected from the probe. With use of such presently disclosed exemplary embodiment, an operator can manipulate the probe for ultrasonic scanning of a practice target aided by visually observing the illumination of the practice target by the projected visible light.

For some such embodiments, such guide light device may project visible color laser light, aligned with the direction of invisible ultrasound waves. For others thereof, such guide light device may comprise a line laser built in to such ultrasound device probe.

For yet other alternatives, such guide light device may comprise a line laser attached to such ultrasound device probe. In some such instances, such device may further comprise a flexible elastic band holder for attaching said line laser to such ultrasound device probe.

In yet other alternatives of the foregoing exemplary embodiment, such practice target may comprise a phantom model of at least one of anatomical and non-anatomical structures embedded in a gel material. In some such instances, such phantom model may comprise one of replicas of normal anatomical structures, actual pathological specimens, and 3D replicas of pathological specimens, to facilitate training operators in how to identify pathology in the structures. In alternatives of the foregoing, such non-anatomical structures may comprise one of selected geometrical shapes of selected colors inserted into a gel for the operator being trained to practice scanning. For other variations of the foregoing, such structures embedded in a gel material may be one of rigid materials fully reflecting ultrasound waves without penetration and gel-like material of variable density and impedance that reflect a portion of the ultrasound waves to give an identifiable ultrasound image with a portion of the ultrasound waves to penetrate beyond the structure to allow deeper structures to also reflect the waves to give an ultrasound image effect similar to that in human tissue.

In other alternatives of the foregoing, such structures may be embedded in a gel material with variable density and impedance to produce ultrasound waves that return to such ultrasound device probe and produce images on a screen visible to an operator that mimic typical ultrasound artifacts of human scanning for image interpretation. In some such variations, such ultrasound artifacts comprise shadowing, posterior enhancement, edge effect, reverberation, and B-lines.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

### BRIEF DESCRIPTION OF THE DRAWINGS

A full and enabling disclosure of the presently disclosed subject matter, including the best mode thereof, directed to one of ordinary skill in the art, is set forth in the specification, which makes reference to the appended Figs., in which:

FIG. 1A shows a representative combination of a presently disclosed probe, laser emitter, and phantom;

FIG. 1B illustrates an ultrasound image of the combination of FIG. 1A;

FIG. 2A illustrates another representative combination of a presently disclosed probe, laser emitter, and phantom;

FIG. 2B illustrates an ultrasound image of the combination of FIG. 2A;

FIG. 3A illustrates another representative combination of a presently disclosed probe, laser emitter, and phantom;

FIG. 3B illustrates an ultrasound image of the combination of FIG. 3A;

FIG. 4A illustrates a generally top and forward perspective view of a representative combination of a presently disclosed probe and attachable laser component, with such laser comprising a line laser mounted on such ultrasonic probe;

FIG. 4B illustrates a generally top and forward perspective view of the representative combination of a presently disclosed probe and attachable laser component of present FIG. 4A, with such laser comprising a line laser and mounting support device separated from such ultrasonic probe;

FIG. 4C illustrates a generally front elevational view of the representative combination of a presently disclosed probe and attachable laser component of present FIG. 4A, with such laser mounted on such ultrasonic probe, and with both resting on a representative support surface;

FIG. 5A illustrates another representative combination of a presently disclosed probe, laser emitter, and phantom with exemplary embedded triangular objects for teaching and practice; and

FIG. 5B illustrates an exemplary ultrasound image obtained from an ultrasound beam and laser line interacting with an exemplary embedded triangular object of application FIG. 5A.

Repeat use of reference characters in the present specification and drawings is intended to represent the same or analogous features or elements of the presently disclosed subject matter.

### DETAILED DESCRIPTION OF THE PRESENTLY DISCLOSED SUBJECT MATTER

Reference now will be made to the embodiments of the presently disclosed subject matter, one or more examples of which are set forth below. Each example is provided by way of an explanation of the presently disclosed subject matter, not as a limitation of the presently disclosed subject matter. In fact, it will be apparent to those skilled in the art that various modifications and variations can be made in the presently disclosed subject matter without departing from the scope or spirit of the presently disclosed subject matter. For instance, features illustrated or described as one embodiment can be used on another embodiment to yield still a further embodiment. Thus, it is intended that the presently disclosed subject matter cover such modifications and variations as come within the scope of the appended claims and their equivalents. It is to be understood by one of ordinary skill in the art that the present discussion is a description of exemplary embodiments only, and is not intended as limiting the broader aspects of the presently disclosed subject matter, which broader aspects are embodied exemplary constructions.

An ultrasound probe is generally provided, either integrally including the presently disclosed subject matter or retrofit therewith. The presently disclosed subject matter preferably will use a visible color laser beam originating from the ultrasound probe itself (when integrally included) or from a laser light pointer attached to an ultrasound probe (when retrofit). In either instance, the visible color laser beam is selected to be able to penetrate a clear-gel phantom. The laser light also preferably will be aligned with the direction of flow of the invisible ultrasound waves so that the learner will know where the ultrasound waves are hitting the anatomical target within the phantom gel.

Providing such immediate visual feedback from the laser light to the learner or operator will inform the learner on how small movements of the probe affect the direction of the ultrasound waves and the quality of ultrasound image obtained. Therefore, practice of such methodology enhances the learner's scanning skill. The presence of the visible laser beam also allows an associated instructor to point out various aspects of the anatomic structures as the laser light strikes them. Thus, the learner not only acquires ultrasound skills but also learns anatomy.

In addition to normal anatomical structures being placed in the phantom gel, real pathological specimens, and 3D replicas of pathological specimens can also be embedded in the gel to assist operators to learn how to scan to identify pathology in the structures such as fractures of a bone. Learners can also practice ultrasound guided procedures with these pathological structures such as withdrawing fluid from a knee with an effusion from infection.

In addition to anatomical structures, structures of various geometrical shapes such as spheres, cones, donuts, etc. can be inserted into the gel to practice scanning. Learners can observe how the ultrasound wave is hitting such structures with the line laser and note changes in the ultrasound image produced on the ultrasound screen with probe manipulation. Such non-anatomical structures can be colored for easy visualization in the gel. They also can be devised to produce a different reflective ultrasound wave based on their composition to create an accurate reflective ultrasound image on the ultrasound screen.

Both anatomical structures and geometrical structures can be composed of rigid material that totally reflect the ultrasound waves without penetrating the structure or composed of gel-like material of variable density and impedance that reflect a portion of the ultrasound waves to give an identifiable ultrasound image, while also allowing a portion of the ultrasound waves to penetrate beyond the structure to allow deeper structures to also reflect the waves to give an ultrasound image of such deeper structures as well. Such reflection characteristics result in ultrasound image effects similar to that in human tissue. Likewise, such material will also allow the laser line to penetrate the structures to show the direction of the ultrasound waves through the structures and deep into the gel to reach other important structures.

3D printing or other production techniques may also advantageously be used to produce laser holders based on the size and shape of existing ultrasound probes that will allow line lasers to be attached to ultrasound probes that do not have the line laser built into the probe itself. In addition, flexible elastic band laser holders can be used for some embodiments to adapt to various sized and shaped ultrasound probes.

Using laser light or any other method to allow a learner using a phantom model to know exactly where the ultrasound beam is being projected is not presently available. Laser light that can easily be seen penetrating clear gel is added by practice of presently disclosed subject matter to an ultrasound probe, to show the direction of the invisible ultrasound waves as they come out of the ultrasound probe. The return or echo of the ultrasound waves to the probe after they hit an object in their path is what produces the image on the ultrasound screen. In learning ultrasound scanning practices, phantom or gel models with human anatomy-like structures such as latex tubing for a blood vessel are embedded in the gel and used to help learners develop the skill of manipulation of the ultrasound probe to obtain quality ultrasound images. Adding a laser light in the same direction as the ultrasound waves per presently disclosed subject matter gives the learner immediate visual feedback as to whether the anatomy-like structure is being adequately struck by the ultrasound waves to produce a quality ultrasound image. Such presently disclosed methodology enhances the learner's ability to acquire effective ultrasound scanning skills and also learn anatomy. 3D printing maybe used to create real-life replicas of both normal and pathological structures to embed in the gel to enhance the learning experience.

Materials of various density and impedance to ultrasound waves can also be used in making the embedded structures to enhance the human tissue fidelity of the ultrasound image produced on the screen. Likewise, the density and the impedance of the gel in which the structures are embedded can be varied to produced waves that return to the probe and produce images on the screen that mimic typical ultrasound artifacts of human scanning such as shadowing, posterior enhancement, edge effect, reverberation, “B” lines, and other artifacts.

The presently disclosed subject is a particular approach to teaching ultrasound which is unique in that there are presently no ultrasound manufactures using laser light or any other wave direction systems for ultrasound teaching.

The presently disclosed subject matter will be a unique ultrasound teaching tool that will give a significant advantage to both ultrasound manufactures as well as simulation/phantom companies. The laser component built into new probes or attached to existing probes will be an advantage to ultrasound systems manufacturers as it will allow their ultrasound machines to be used with the clear phantom to allow buyers of such technology to more easily learn ultrasound.

Additionally, per the presently disclosed methodology, the laser component can be turned off or taken off of the probe if it is not being used for learning purposes with a phantom trainer. Thus, it will be economical to include the presently disclosed technology either within or attached to the ultrasound probe when compared relative to the market value it will add to such an ultrasound machine.

Simulation/phantom companies will find that the presently disclosed methodology of teaching ultrasound gives them an advantage over those promoting only traditional methods and thus result in an expansion of their market share. One of the most commonly used applications of ultrasound today is the ultrasound-guided procedure applications. Ultrasound-guided catheter placement into blood vessels and ultrasound-guided placement of medication into joints are among the most common daily applications of ultrasound. The presently disclosed subject matter is ideal for helping learners develop skill and competency in performing such high-demand ultrasound-guided procedures.

For those ultrasound manufacturers who incorporate the presently disclosed ultrasound laser directional system into their ultrasound units would have a market advantage in that the same ultrasound systems they are selling could be used for both learning and then professional practice of ultrasound. Also, learners tend to buy equipment for their professional clinical practice of ultrasound equipment that they have learned on and know well.

It would also be advantageous in the simulation center market that is teaching ultrasound procedures. Buying an ultrasound system or attachable laser light component that includes a laser directional system would add flexibility to the ultrasound systems used in such a simulation center.

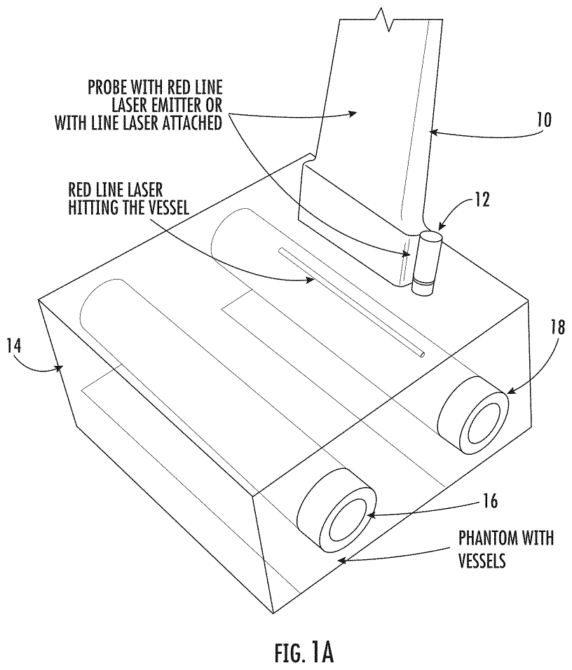

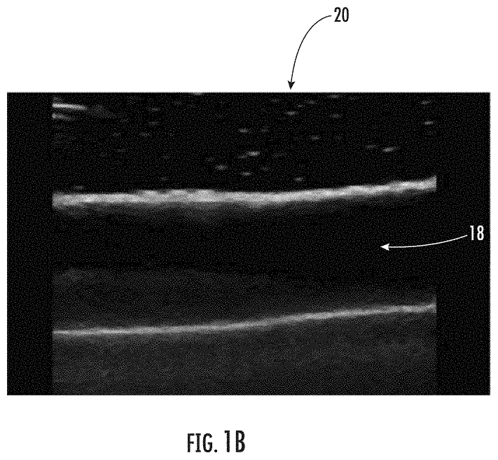

FIG. 1A shows a representative combination of a presently disclosed probe, laser emitter, and phantom, with FIG. 1B illustrating an ultrasound image of the exemplary combination of FIG. 1A. More specifically, an ultrasound probe generally 10 has a laser emitter or line laser generally 12 attached. As shown, a preferably colored laser line (in this instance, red) is produced in alignment with the ultrasound transmissions of probe 10. Exemplary representative phantom generally 14 comprises an at least partially transparent gel material, to visibly show to an operator in training representative vessels 16 and 18.

FIG. 1B represents that vessel 18 appears very clearly on the monitoring screen 20 because in the example of FIG. 1A, the ultrasound waves are aligned for perfectly striking the vessel 18 as desired (as shown by the projected red laser line on vessel 18 in FIG. 1A). Stated another way, an operator in training (not shown) may impart manual movement to probe 10 relative to phantom 14 until visually cued for proper alignment of the laser light from laser 12 onto vessel 18 within phantom 14, all as shown by the ultrasound image illustrated by display 20.

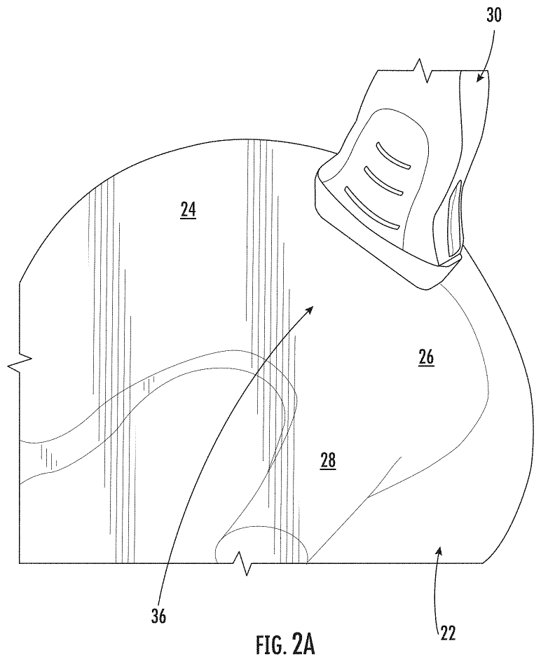

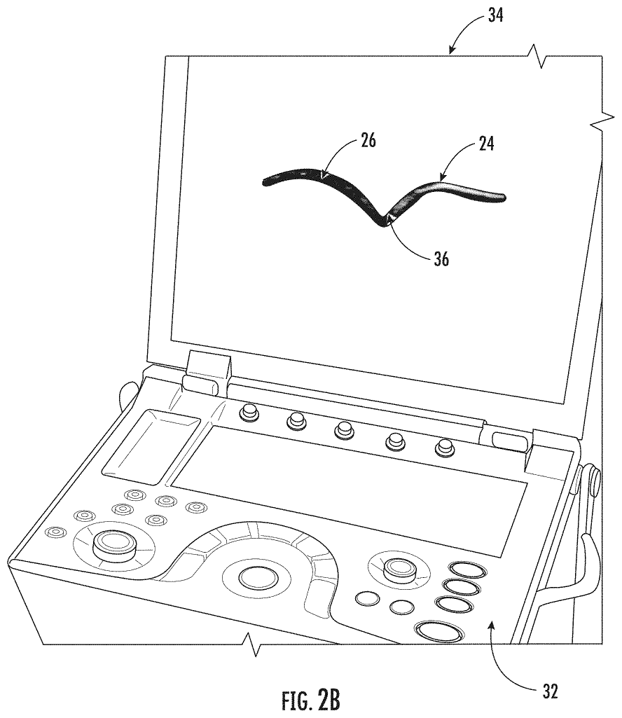

FIG. 2A illustrates another representative combination of a presently disclosed probe, laser emitter, and phantom, while FIG. 2B illustrates an ultrasound image of such combination of FIG. 2A. In the presently disclosed subject matter represented by FIGS. 2A and 2B, a phantom generally 22 provides a model of a human shoulder joint consisting of the scapula generally 24 and the head generally 26 of the humerus 28 embedded in a polymer gel. A red line laser pointer was then used with probe 30 to identify the path of the ultrasound waves as they traversed the gel and bounced off the model and back to the ultrasound probe 30 of ultrasound device generally 32 to be converted into an ultrasound image generally 34. Thus, learners can develop their skills in capturing a good ultrasound image of the shoulder while noting the relationship of the humerus to the scapula in a human shoulder. They can also identify the shoulder joint space 36 where medication could be directed with ultrasound if needed for treatment.

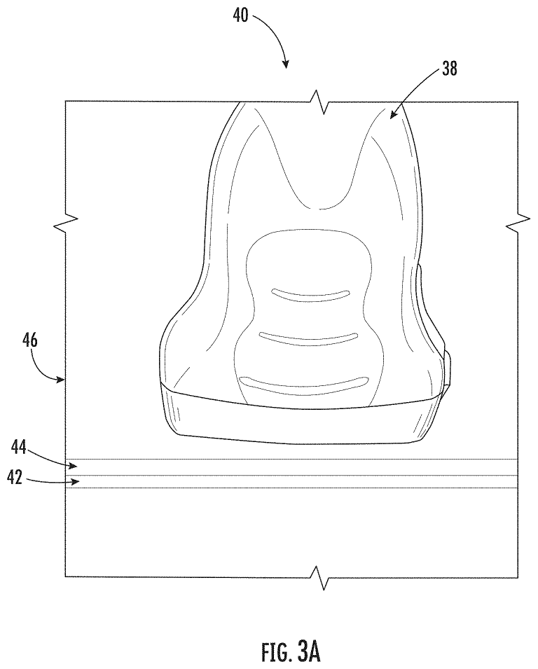

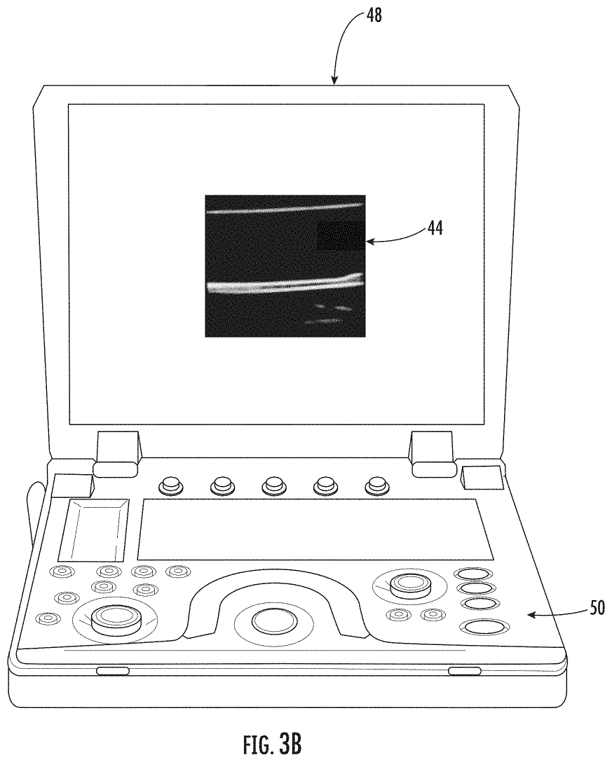

FIG. 3A illustrates another representative combination of a presently disclosed probe, laser emitter, and phantom, with FIG. 3B illustrating an ultrasound image of the combination of FIG. 3A. In particular, probe generally 38 is guided by an operator's hand generally 40 such that a projecting line laser generally 42 is aligned with a vessel (latex tubing 44) within transparent (or clear) phantom generally 46. As represented by FIG. 3B, an image generally 48 is created on the display of ultrasound device 50, showing an image of the latex tubing 44 perfectly on the screen because the operator 40 has used the laser line 42 with the ultrasound beam from probe 38 to accurately align the probe 38 relative to the phantom vessel tubing 44.

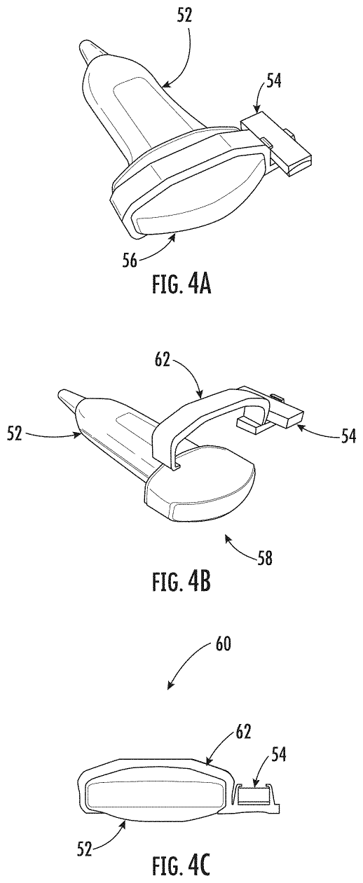

FIG. 4A illustrates a generally top and forward perspective view of a representative combination of a presently disclosed probe generally 52 and attachable laser component generally 54, with such laser comprising a line laser mounted on such ultrasonic probe. As understood by those of ordinary skill in the art, curved face generally 56 of probe 52 both produces and receives ultrasonic waves as returned (“bounced back”) from a target. When properly mounted, line laser 54 generates an expanded line 58 in plane 60, which is in alignment with the ultrasonic patterns emitted from face 56 of probe 52.

FIG. 4B illustrates a generally top and forward perspective view of the representative combination of a presently disclosed probe 52 and attachable laser component 54 of present FIG. 4A, with such laser comprising a line laser and mounting support device generally 62, which is shown separated from such ultrasonic probe 52. Such device 62 may be variously formed, for example, from elastic or resilient material to in effect “grip” mount onto probe 52 and similarly receive laser 54 in a resilient interference fit.

FIG. 4C illustrates a generally front elevational view of the representative combination of a presently disclosed probe 52 and attachable laser component 54 of present FIG. 4A, with such laser mounted on such ultrasonic probe, and with both associated with a representative support device 62 (shown as an adapter in a “snap-on” type of arrangement). The exemplary arrangements of FIGS. 4A through 4C are particularly useful with ultrasound probes that do not have a built-in line laser. As will be understood by those of ordinary skill in the art from the complete disclosure herewith, laser 54 is perfectly aligned with ultrasonic waves produced by probe 52, whenever such laser is properly mounted with support device 62.

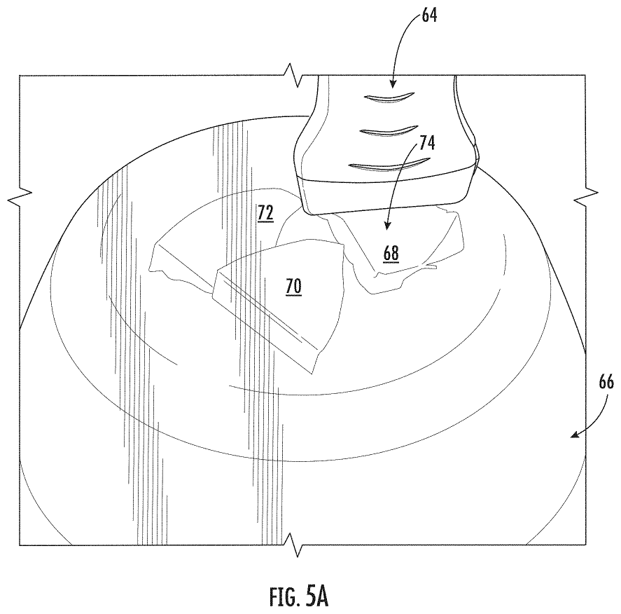

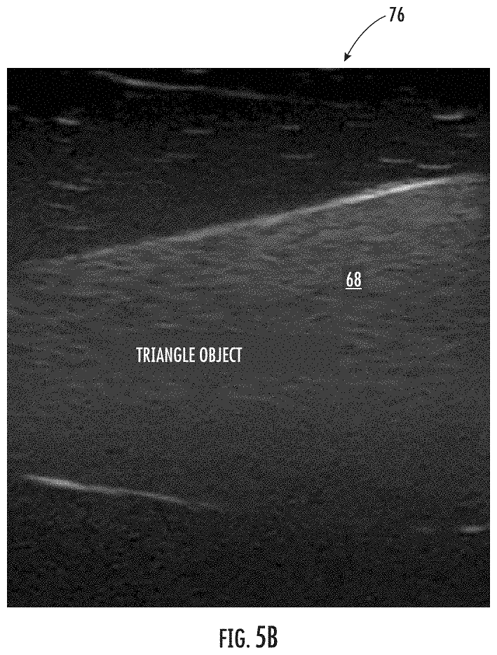

As otherwise discussed herein, some phantoms per presently disclosed methodology may for practice and instruction include various embedded geometrical shapes. FIG. 5A illustrates another representative combination of a presently disclosed probe generally 64 with laser emitter (not seen), and phantom generally 66 with exemplary embedded triangular objects 68, 70, and 72 for teaching and practice. While the laser emitter is not seen in the view of FIG. 5A, the emitted laser line 74 is represented as engaged with object 68.

FIG. 5B illustrates an exemplary ultrasound image generally 76 obtained from an ultrasound beam and laser line interacting with an exemplary embedded triangular object 68 of application FIG. 5A. Again, manipulation of probe 64 by an operator in training, in association with projected laser line 74, allows such operator to use the image 76 to gain knowledge and experience in how directing probe 64 relates to the resulting image 76.

These and other modifications and variations to the presently disclosed subject matter may be practiced by those of ordinary skill in the art, without departing from the spirit and scope of the presently disclosed subject matter, which is more particularly set forth in the appended claims. In addition, it should be understood the aspects of the various embodiments may be interchanged both in-whole or in part. Furthermore, those of ordinary skill in the art will appreciate that the foregoing description is by way of example only, and is not intended to limit the presently disclosed subject matter so further described in the appended claims.

What is claimed is:

1. A method for training an operator to use an ultrasound device, comprising:
providing an ultrasound device having a probe which can be manipulated by an operator relative to a practice target, with such probe selectively projecting ultrasound waves; and
associating with such probe a guide light device configured to project visible light in a projection area which coincides with that of ultrasound waves projected from the probe, whereby an operator can manipulate the probe for ultrasonic scanning of a practice target aided by visually observing the illumination of the practice target by the projected visible light.

2. A method as in claim 1, wherein said guide light device projects visible color laser light, aligned with the direction of invisible ultrasound waves.

3. A method as in claim 2, wherein said guide light device comprises a line laser built in to said ultrasound device probe.

4. A method as in claim 2, wherein said guide light device comprises a line laser attached to said ultrasound device probe.

5. A method as in claim 4, wherein said line laser is attached to said ultrasound device probe using a flexible elastic band holder conforming to the shape of said ultrasound device probe.

6. A method as in claims 4, further comprising using 3D printing to produce a laser holder for attachment of said line laser to said ultrasound device probe.

7. A method as in claim 1, wherein said practice target comprises a phantom model embedded in a gel material.

8. A method as in claim 7, wherein:
said phantom model represents human anatomy-like structures; and
said gel material is transparent to light.

9. A method as in claim 8, wherein said human anatomy-like structures comprise one of bone, joint, and latex tubing for a blood vessel.

10. A method as in claim 8, wherein said human anatomy-like structures comprise one of human tissue bone, joint, vessels, blood, fat, muscle, tendon, nerves, skin and organs.

11. A method as in claim 7, wherein said phantom model comprises one of replicas of normal anatomical structures, actual pathological specimens, and 3D replicas of pathological specimens, to facilitate training operators in how to identify pathology in the structures.

12. A method as in claim 1, wherein said practice target comprises a phantom model of at least one of anatomical and non-anatomical structures embedded in a gel material.

13. A method as in claim 12, wherein said non-anatomical structures comprise one of selected geometrical shapes of selected colors inserted into a gel for the operator being trained to practice scanning.

14. A method as in claim 12, wherein said structures embedded in a gel material may be one of rigid materials fully reflecting ultrasound waves without penetration or gel-like material of variable density and impedance that reflect a portion of the ultrasound waves to give an identifiable ultrasound image with a portion of the ultrasound waves to penetrate beyond the structure to allow deeper structures to also reflect the waves to give an ultrasound image effect similar to that in human tissue.

15. A method of operator training, using an ultrasound device, comprising:
providing an ultrasound device having an associated screen visible to an operator; and
associating a guide light with the ultrasound device so that the operator can visually observe light indicating the exact direction and anatomy of contact points of ultrasound waves emanating from said ultrasound device.

16. A method as in claim 15, further comprising associating a practice target with said ultrasound device, such that the operator can manipulate the ultrasound device for ultrasonic scanning of such practice target aided by visually observing the illumination of the practice target by the projected visible light.

17. A method as in claims 16, wherein said projected visible light comprises laser light from a laser device associated with said ultrasound device.

18. A method as in claim 17, wherein:
said ultrasound device has a probe manipulated by an operator; and
said probe has a laser light formed therewith or attached thereto.

19. A method as in claim 18, wherein said laser light comprises the output of a line laser.

20. An ultrasound device with directional light for 3D anatomy ultrasound phantom trainer, comprising:
an ultrasound device having a probe which can be manipulated by an operator relative to a practice target, with such probe selectively projecting ultrasound waves; and
a guide light device associated with said probe, and configured to project visible light in a projection area which coincides with that of ultrasound waves projected from the probe, whereby an operator can manipulate the probe for ultrasonic scanning of a practice target aided by visually observing the illumination of the practice target by the projected visible light.

21. A device as in claim 20, wherein said guide light device projects visible color laser light, aligned with the direction of invisible ultrasound waves.

22. A device as in claim 21, wherein said guide light device comprises a line laser built in to said ultrasound device probe.

23. A device as in claim 21, wherein said guide light device comprises a line laser attached to said ultrasound device probe.

24. A device as in claim 23, further comprising a flexible elastic band holder for attaching said line laser to said ultrasound device probe.

25. A device as in claim 20, wherein said practice target comprises a phantom model of at least one of anatomical and non-anatomical structures embedded in a gel material.

26. A device as in claim 25, wherein said phantom model comprises one of replicas of normal anatomical structures, actual pathological specimens, and 3D replicas of pathological specimens, to facilitate training operators in how to identify pathology in the structures.

27. A device as in claim 25, wherein said non-anatomical structures comprise one of selected geometrical shapes of selected colors inserted into a gel for the operator being trained to practice scanning.

28. A device as in claim 25, wherein said structures embedded in a gel material may be one of rigid materials fully reflecting ultrasound waves without penetration and gel-like material of variable density and impedance that reflect a portion of the ultrasound waves to give an identifiable ultrasound image with a portion of the ultrasound waves to penetrate beyond the structure to allow deeper structures to also reflect the waves to give an ultrasound image effect similar to that in human tissue.

29. A device as in claim 25, wherein said structures are embedded in a gel material with variable density and impedance to produce ultrasound waves that return to said ultrasound device probe and produce images on a screen visible to an operator that mimic typical ultrasound artifacts of human scanning for image interpretation.

30. A device as in claim 29, wherein said ultrasound artifacts comprise shadowing, posterior enhancement, edge effect, reverberation, and B-lines.
